(19) United States
(12) Patent Application Publication
(10) Pub. No.: US 2006/0211743 A1
(43) Pub. Date: Sep. 21, 2006

(54) 3-Pyridinecarboxamide reduces nerve degeneration

(75) Inventors: Zhigang He, Boston, MA (US); Jing Wang, Boston, MA (US); Ying Chen, Boston, MA (US); Shijniro Kaneko, Boston, MA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(73) Assignee: Children's Medical Center Corporation

(21) Appl. No.: 11/085,948

(22) Filed: Mar. 21, 2005

(51) Int. Cl. A61K 31/455; G06Q 10/00

(52) U.S. Cl. 514/356; 705/2

(57) ABSTRACT

Neural degeneration is reduced in a patient determined to be suffering from chronic neurodegeneration by administering to the patient an effective amount of 3-pyridinecarboxamide (nicotinamide) and detecting a resultant decrease in the neural degeneration.

This work was supported by Federal Grant No. R01-NS41044 from NIH. The government may have rights in any patent issuing on this application.

### FIELD OF THE INVENTION

The invention is in the field of using high chronic doses of 3-pyridinecarboxamide to reduce nerve degeneration.

### BACKGROUND OF THE INVENTION

Behavioral studies in aging have demonstrated a natural progression of cognitive decline over the adult lifespan, with such decline being more accelerated in individuals with neurodegenerative dementia such as Alzheimer's diseases (Gallagher and Rapp, 1997, Hedden and Gabrieli, 2004). Morphologically, the phenomenon of the aging brain has traditionally been characterized in terms of such classical traits as neuronal death, neuronal atrophy (including synaptic loss with dendritic and axonal degeneration), and aging pigments (lipofuscins). Until recently it was widely accepted that neuronal death was an inevitable result of normal aging. Yet, with the development of more accurate methods for counting neurons (West and Gundersen, 1990; West et al., 1994), it has been shown that the neuronal atrophy characterized by synaptic loss and the degeneration of dendrites and axons correlates more closely with cognitive decline, and often occur earlier and to a greater extent than the death of the neuronal perikarya (reviewed by Morrison and Hof, 1997; Peters, 2002, Hof and Morrison, 2004). For example, from rodents to humans, dendritic arbor, spine and synaptic morphology have all been reported to undergo age-related regressive changes which could impact upon the function of hippocampal circuits but would not be reflected as neuronal death (Coleman and Flood, 1987). Similar changes also occur in other regions of the nervous system (Anderson and Rutledge, 1996, Jacobs et al., 1997, Chen and Hillmanm 1999, Duan et al., 2003). In addition, these dendritic and synaptic changes are accompanied by degeneration of myelinated axons in the deep layers of the cortex and in white matter, which correlates in old animals with deficits in visual and spatial recognition tasks (Peters et al., 2000, Sandell and Peters, 2003). Such degenerative processes appear to be more accelerated in the brains of Alzheimer's patients where neuronal death often becomes obvious (Walsh and Selkoe, 2004). Because of its frequent occurrence in the absence of neuronal death, neuronal atrophy may result from an apoptosis-independent molecular mechanism that remains unknown.

A potentially useful model for studying the degenerative mechanism of neuronal processes and synaptic connections is the self-destructive process observed at the distal portion of a transected axon after injury, termed “Wallerian degeneration” (Waller, 1850; Raff et al., 2002; Coleman and Perry, 2002; Gillingwater and Ribchester, 2003). Studies in rodents have established that degeneration of distal axons occurs within 24-48 hr after axotomy, and is often preceded by the loss of neuromuscular junctions and other types of synapses by several hours (Miledi & Slater, 1969, 1970; Winlow and Usherwood, 1975). Similar to age-related synaptic and neuronal process degeneration, Wallerian degeneration has been proposed to operate via an apoptosis-independent program (Raff et al., 2002). Such degenerative processes do not seem to involve activation of the caspase family cysteine proteases (Finn et al., 2000, Sievers et al., 2003). In addition, Wallerian degeneration can be mechanistically separated from the apoptosis triggered by NGF deprivation in sympathetic neurons (Deckwerth and Johnson, 1994, Bume et al., 1996, Zhai et al., 2003).

Initial insights into the molecular mechanisms of Wallerian degeneration came with the discovery of a spontaneously occurring mutant mouse strain, C57BL/Wlds, whose axons and synapses survived for as long as weeks after transection (Lunn et al., 1989, Glass et al., 1993). Interestingly, the slow Wallerian degeneration phenotype appears only in young Wlds mice of up to 4-7 months of age (Tsao et al., 1994, Gillingwater et al., 2002), suggesting that the Wlds-dependent axon protection might be modulated by age-dependent mechanisms. Genetic studies have attributed this slow Wallerian degeneration phenotype to the over-expression of a fusion protein (Wlds) which consists of the full-length nicotinamide mononucleotide adenylyltransferase (Nmnat1), an enzyme required for both the de novo and salvage pathways of nicotinamide adenine dinucleotide (NAD) biosynthesis (Schweiger et al., 2001, Garavaglia et al., 2002), and a short region of a ubiquitin assembly protein UFD2 (Koegl et al., 1999) (Conforti et al., 2000, Mack et al., 2001). These results suggest the possible involvement of the ubiquitin-proteasome system (UPS) and/or the NAD-synthesizing enzyme Nmnat1 in the process of Wallerian degeneration. By using in vitro cultured neurons, it has been shown that inhibiting UPS activity can slow down Wallerian degeneration (Zhai et al., 2003). Similarly, a recent structure and function study demonstrated that over-expressing Nmnat1 alone could prevent the axon degeneration triggered by mechanical or chemical insults in cultured neurons (Araki et al., 2004), substantiating the notion that overexpression of Nmnat1 may prevent transection-triggered axon degeneration. In addition, this study further suggested that the protective effect of Nmnat1 is mediated by an NAD-dependent nuclear deacetylase Sirt1 (Araki et al., 2004), a mammalian homologue of yeast sir2 which has been implicated in regulating lifespan in yeast (reviewed by Lin and Guarante, 2003). However, previous studies revealed that NAD levels are not increased in the tissues of Wlds mice (Mack et al., 2001). These studies raise several important questions: Whether and how are NAD and/or Sirt1 involved in axon degeneration? Is there a common mechanism for axon degeneration and other aging-related degenerative processes in mammalian neurons such as dendritic degeneration and synaptic loss?

Here we demonstrate that NAD decrease is common to aging-related degenerative processes in neurons, including axon degeneration as well as dendrite degeneration and synaptic loss. Instead of acting through the nuclear Sirt1, NAD affects local energy metabolism. Together with our finding of a progressive decrease in NAD levels in more vulnerable regions of the mouse brain over their adult lifespan, we disclose that impaired NAD and energy metabolism is an important mechanism underlying the degenerative process in aging and degenerative brains, and that high dose 3-pyridinecarboxamide (nicotinamide) can be used to reduce nerve degeneration associated with neurodegenerative disease.

Relevant Literature: Adams et al. (U.S. Pat. No. 5,736,529); Rainer (J Neural Transm 2000; 107, 1475-81); Araki et al. (2004, Science 305, 1010-1013).

### SUMMARY OF THE INVENTION

One aspect of the invention is a method for reducing neural degeneration in a patient determined to be suffering from chronic neurodegeneration, the method comprising the steps of: administering to the patient an effective amount of 3-pyridinecarboxamide (nicotinamide); and detecting a resultant decrease in the neural degeneration; wherein the effective amount is 1 to 40 g/day and is administered daily for at least 90 days.

In one embodiment of the invention, the patient is determined to be suffering from a neural degenerative disease or disorder selected from Alzheimer's Disease, Huntington's Disease, Parkinson's Disease, amyotrophic lateral sclerosis (ALS), macular degeneration, and progressive supranuclear palsy.

In certain embodiments, the effective amount is 1 to 20 g/day, or 2-10 g/day.

In certain embodiments, the nicotinamide is administered daily for at least 6 months, or at least 12 months.

In one embodiment of the invention, the nicotinamide is administered orally.

Another aspect of the invention is a method of doing business comprising promoting, marketing, selling or licensing a method for reducing neural degeneration in a patient determined to be suffering from chronic neurodegeneration, wherein the method for reducing neural degeneration in a patient comprises the steps of: administering to the patient an effective amount of 3-pyridinecarboxamide (nicotinamide), and detecting a resultant decrease in the neural degeneration; wherein the effective amount is 1 to 40 g/day and is administered daily for at least 90 days.

### DETAILED DESCRIPTION OF SPECIFIC EMBODIMENTS OF THE INVENTION

The invention provides a method for reducing neural degeneration in a patient determined to be suffering from chronic neurodegeneration. The method comprises the steps of chronically administering to the patient an effective amount of 3-pyridinecarboxamide (nicotinamide), and detecting a resultant decrease in the neural degeneration.

The patient can be any mammalian animal suffering from chronic neurodegeneration, including pets, livestock, and animal models of human disease (e.g. rodent, canine, and primate models). In particular embodiments, the patient is a human.

The patient is determined to be suffering from chronic neurodegeneration, typically based on the presence of a characteristic clinical history and neurologic findings for the particular neural degenerative disease or disorder. Diagnosis of a particular neural degenerative disease is made in accordance with practice guidelines published by the American Academy of Neurology (ANN). In one embodiment of the invention, the patient is a human who has been determined to be suffering from a neural degenerative disease or disorder selected from Alzheimer's Disease, Huntington's Disease, Parkinson's Disease, amyotrophic lateral sclerosis (ALS), macular degeneration, and progressive supranuclear palsy.

The effective amount of nicotinamide is in the range of about 5, 10, 15 or 20 to about 50, 100, 250 or 500 mg/kg body weight (bw)/day, and preferably in the range of 10 to 250, 15 to 100, or 15 to 50 mg/kg bw/day. For human patients, preferred effective amounts are in the range of about 1 to about 5, 10, 20 or 40 g/day.

The nicotinamide is administered chronically, i.e. regularly over a period of at least 3, preferably at least 6, more preferably at least 12 months, wherein the regularity is at least once, preferably at least 2-3 times, more preferably at least 7 times (i.e. daily) per week. In one embodiment of the invention, the effective amount is 1-40 g/day and is administered daily over at least 90 days, preferably at least 6 months, and more preferably at least 1 year.

The nicotinamide can be administered by any conventional method such as injection, transdermal patch, oral administration, etc. For ease of use and patient compliance, the nicotinamide is preferably administered orally. The oral dosages can be in any suitable form, including tablets, capsules, lozenges, troches, hard candies, powders, metered sprays, creams, suppositories, syrups, etc. In one embodiment, the nicotinamide is administered in tablet form, either as an immediate release or sustained-release formulation. The nicotinamide is combined with a pharmaceutically acceptable excipient such as gelatin, an oil, etc. and may include additional active agents.

The optimal amount and applicable protocols for administration of nicotinamide for a particular neural degenerative disease or disorder are readily derived from clinical trials of nicotinamide for the treatment of a particular neural degenerative disease or disorder.

In the detecting step, a resultant decrease in neural degeneration is detected. Decreases in neural degeneration can be detected directly by art-known imaging methodology such as diffusion tensor imaging or magnetic resonance imaging. Alternatively, decreases in neural degeneration can be detected inferentially, for example, by a reduction in biological markers for the disease or disorder, amelioration of neurological deficits, etc.

Another aspect of the invention is a method of doing business comprising promoting, marketing, selling or licensing any of the aforementioned methods for reducing neural degeneration in a patient determined to be suffering from chronic neurodegeneration. In one embodiment, the method that is promoted, marketed, sold, or licensed comprises the steps of: administering to the patient an effective amount of 3-pyridinecarboxamide (nicotinamide); and detecting a resultant decrease in the neural degeneration; wherein the effective amount is 1 to 40 g/day and is administered daily for at least 90 days.

### EXAMPLE 1

### NAD Decreases in Degenerating Axons

Previous studies have demonstrated that despite increased Nmnat1 activity, NAD levels are not increased in tissues of Wlds mice (Mack et al., 2001) raising the question of whether NAD or its derivatives are the effector molecules in preventing Wallerian degeneration. As a first step in examining the role of NAD in Wallerian degeneration, we used an ultra-sensitive high-performance liquid chromatography (HPLC) method (Micheli and Sestini, 1997) to measure the concentration of NAD and its derivatives in degenerating axons. To do this, the acidic lysates were prepared from axonal segments collected at different time points after transecting the axons from cultured dorsal root ganglia (DRG) and resolved by reverse-phase chromatography. 2 hr after axotomy, when the transected axons were still morphologically indistinguishable from their uncut controls, NAD levels began to decrease. This NAD decrease continued over the course of degeneration, reaching about 50% at 4 hr post-axotomy, to almost undetectable levels by 6 hr after axotomy, when early morphological signs of degeneration began to develop. The NAD decline observed is not due to leakage across the axonal membrane, as other small molecules, such as inosine 5-monophosphate (IMP), AMP, nicotinic acid (Na) and GDP, did not decrease in the degenerating axons. By a similar approach of alkaline extraction (Micheli and Sestini, 1997), it has been difficult for us to detect NADH in the axonal extracts, presumably due to the low abundance of NADH and the high NAD/NADH ratio reported in mammalian cells (Zhang et al., 2002, Kasischke et al., 2004, Kannurpatti et al., 2004). A similar NAD decline was also observed in vincristine-treated DRG neurons, another commonly used axon degeneration model (Wang et al., 200 lb, Araki et al., 2004). These results together directly demonstrate a decrease in axonal NAD levels during degeneration.

### EXAMPLE 2

### Wlds and Nmnat1 Prevent the Decrease of NAD Levels in Transected Axons

We next examined whether over-expression of Wlds or Nmnat1 could prevent the NAD decrease in degenerating axons. To do this, we made recombinant herpes simplex viruses (HSV) to express either Wlds fusion protein or the full-length Nmnat1 in cultured neurons. Consistent with a recent study (Araki et al., 2004), we found that both the Wlds and Nmnat1 proteins could significantly slow down the degenerative process of transected axonal segments in cultured DRG neurons. Neither Wlds nor Nmnat1 significantly increased neuronal NAD levels in intact neurons. Yet, over-expression of either protein profoundly delayed the NAD decrease in the transected axons in a temporal pattern similar to their protective effects on axonal degeneration. The ability of Nmnat1 and Wlds over-expression in preventing NAD decrease in transected axonal segments indicates that these over-expressed proteins may enhance the NAD-synthesizing activity in the transected axons. Consistently, we observed that the over-expressed Wlds and Nmnat1 proteins were present not only in the nuclei of DRG neurons, but also in the axons as determined by both immunostaining and Western blotting (see also, Wang et al., 2001a). Thus, our results indicate that over-expressed Wlds/Nmnat1 proteins can exert their protective effects by synthesizing NAD locally and preventing NAD decline resulting from transection.

### EXAMPLE 3

### Local Application of NAD Prevents Axon Degeneration

To definitively determine whether preventing the local decrease in NAD levels could mediate axon protection, we employed soma-free axonal segments that have been separated from their soma as models to examine the protective effects of different pharmacological treatments. It has been shown that exogenously provided NAD, when directly added to the culture medium of DRG neurons, can be transported across the axonal membrane (Araki et al., 2004). Thus, we first examined the protective effects of different concentrations of NAD added to the culture before or after axon transection and removal of ganglia. Similar to previous observations (Araki et al., 2004), we found that pre-treating DRG neurons with NAD 24 hr prior to transection delayed axon degeneration. However, different from the concentrations (0.1 to 1 mM) of NAD used by Araki et al (2004), we found that only higher concentrations (1-20 mM) of NAD resulted in significant protective effects. Surprisingly, we found that the same concentrations of NAD also efficiently prevented axon degeneration when added at the time of axon transection, indicating that locally provided NAD is sufficient to prevent axon degeneration.

To further assess the local and nuclear contribution to such an NAD-dependent protection, we compared the protective effects of NAD provided at different time points. We found that pre-treatment with NAD 24 hr prior to transection was similarly protective as adding the same concentrations of NAD at the time of transection or up to 3 hr post-transection. However, at 7 hr after transection when axonal NAD had dropped to undetectable levels, exogenously provided NAD failed to show any significant protective effect. These results indicate that NAD-mediated protection acts primarily through a local mechanism, arguing against a significant contribution of NAD-induced transcription and other nuclear events. In this respect, our results are inconsistent with those shown by Araki et al (2004), possibly due to the different concentrations of NAD used in our studies (1-20 mM) and by Araki et al (0.1 to 1 mM).

Further documenting a local protective effect of NAD, we found that nicotinamide, a precursor of NAD synthesis, had a similar protective effect even when applied at the time of axonal transection or 3 hr post-transection. HPLC analysis indicated that both exogenously applied NAD and nicotinamide delayed the decrease of axonal NAD levels triggered by transection. Together, these results indicate that NAD can affect the process of axon degeneration through a local protective mechanism.

### EXAMPLE 4

### Sirt1 is Not Required for NAD-Mediated Axon Protection

We next attempted to explore the possible mechanisms underlying this local NAD-dependent axon protection. As a recent study showed that RNA interference-mediated Sirt1 gene silencing could block NAD-dependent axonal protection (Araki et al., 2004), we first examined the involvement of Sirt1 in Wallerian degeneration by using neurons derived from Sirt1 (+/+) and Sirt1 (−/−) mice which have been previously characterized (McBurney et al., 2003; Motta et al., 2004; Picard et al., 2004). Surprisingly, we failed to observe any significant difference between Sirt1 (+/+) and Sirt1 (−/−) neurons in transection-triggered axonal degeneration both in the absence or presence of NAD. Moreover, when infected with HSVs expressing Wlds or Nmnat1, axon degeneration was indistinguishably prevented in DRG neurons from Sirt1 (+/+) and Sirt1 (−/−) mice. Together, these results show that Sirt1 is not required for Wlds/Nmnat1-mediated protection from Wallerian degeneration. A possible explanation for this discrepancy between Araki et al (2004) and our results is the different approaches used to block Sirt1 activity. While the RNAi-mediated gene silencing used by Araki et al (2004) could result in acute suppression of Sirt1 expression in cultured neurons, other Sirt family members (sirturins) might be up-regulated in the Sirt1 (−/−) neurons used in our study, thus compensating for the loss of Sirt1 function. It is known that 6 other sirturins exist and some of these proteins are localized in the cytoplasmic compartment (Onyango et al., 2002; North et al., 2003). However, we failed to observe any significant change in the extent of axon degeneration by including a range of concentrations of resveratrol (Howitz et al., 2003), a specific activator of Sirt1 and other sirturins. On the other hand, two known sirturin inhibitors, sirtinol (Grozinger et al., 2001) and nicotinamide (50 μM) (Luo et al., 2001, Bitterman et al., 2002, Anderson et al., 2003, Marcotte et al., 2004), did not attenuate the protective effect of Nmnat1 or NAD.

### EXAMPLE 5

### Inhibition of Glycolysis and ATP Production Mediates Axon Degeneration Triggered by NAD Decrease

In addition to acting as co-factors for Sirt1 and other NAD-dependent enzymes, NAD is also essential for ATP-synthesizing redox reactions which are involved in glycolysis and oxidative phosphorylation (Shibata et al., 1991). In particular, cytosolic NAD is required in the glycolytic pathway for the conversion of glyceraldehyde 3-phosphate to 1,3-bisphosphoglycerate. Thus, upon cytosolic NAD depletion, glucose can no longer be converted to the pyruvate needed to fuel oxidative phosphorylation in mitochondria. Considering the significant energy demand of various physiological functions in axons, such as maintenance of membrane potential and axonal transport, it is conceivable that decreased levels of NAD may impair axonal energy production, thus triggering the onset of axon degeneration. In support of this, we found that axonal ATP levels decreased in parallel with NAD levels by HPLC analysis of degenerating axons. Furthermore, both over-expression of Wlds/Nmnat1 and exogenously providing NAD or nicotinamide delayed the decrease of ATP levels in transected axons that have been separated from their somas.

To further assess whether the glycolytic blockade resulting from cytosolic NAD depletion contributes to axon degeneration, we examined whether the degeneration of axotomized axons could be suppressed by supplying axons with the glycolytic end product pyruvate which can be used directly for oxidative phosphorylation in mitochondria. When added at the time of transection, 10 mM methyl-pyruvate could significantly delay the decline of axonal ATP levels and also protect axons from degeneration. However, the protective effects of methyl-pyruvate became weaker at 24 hr post-transection. Nevertheless, these results indicate that ATP production is an important effector of NAD depletion in axon degeneration.

### EXAMPLE 6

### Decreasing Cellular NAD Levels Induces “Dying-Back” Axon Degeneration in Cultured Neurons

We next examined whether decreasing neuronal NAD levels is sufficient to induce axon degeneration in intact neurons. By sequence alignment, a conserved (T/H)XGH motif has been identified in the N-terminus of all Nmnat proteins from different species (Saridakis et al., 2001). This motif likely represents a critical active site, as a mutant enzyme with a point mutation at the last amino acid (H to A) loses its catalytic activity but retains its ability to bind the nicotinamide mononucleotide (NMN) substrate (Saridakis et al., 2001). Thus, we made a mutant Nmnat1 in which the critical histidine residue at amino acid 24 in this active site motif was substituted with alanine. Since most of the NAD in mammalian cells, except for liver and kidney cells, is synthesized by recycling degraded NAD products such as nicotinamide via the salvage pathway (Shibata et al., 1991, Rongvaux et al., 2003), over-expressing this mutant Nmnat1 would be expected to sequester the limited substrates, thus decreasing cellular NAD levels. As expected, we found that HSV-mediated over-expression of this mutant Nmnat1, but not wild type Nmnat1 or YFP, indeed gradually decreased neuronal NAD levels in superior sympathetic ganglia (SCGs) and other neuronal types, such as DRG and hippocampal neurons. As a result, neurons expressing this mutant Nmnat1 developed signs of axon degeneration in the absence of axotomy, concomitant with decreases in neuronal NAD and ATP levels. Interestingly, the axon degeneration triggered by this mutant Nmnat1-induced NAD decrease started from the distal axonal segments and progressively spread toward the cell body, reminiscent of the “dying-back” phenomenon found in degenerating brains (Peters et al., 2000, Saigoh et al., 1999, Raff et al., 2002).

This degenerative process apparently resulted from the NAD-lowering effect of the mutant Nmnat1 for the following reasons: First, this degeneration did not occur in neurons infected with the same titer of HSV expressing YFP or wild type Nmnat1. Second, it could be prevented by exogenous application of NAD. In addition, methyl-pyruvate can also efficiently prevent the axon degeneration, consistent with the notion of a critical involvement of ATP depletion in the degenerative process. Importantly, the axon degeneration observed is not secondary to neuronal apoptosis as we failed to detect any apoptotic signs by staining with both Hoechst and antibodies against activated caspase 3 in these neurons. In addition, a caspase inhibitor Z-DEVD-fmk also had no effect on the axon degeneration induced by this mutant Nmnat1 expression. Thus, our results indicate that decreasing neuronal NAD level is sufficient to trigger an apoptosis-independent degenerative process that starts from the axonal terminal in cultured neurons.

### EXAMPLE 7

### Decreasing Neuronal NAD Levels Leads to the Synaptic Loss and Process Degeneration in Cultured Hippocampal Neurons

We next attempted to examine whether decreasing NAD levels can induce similar “dying-back” axon degeneration in neurons with established synaptic connections. Such experiments might allow us to assess whether the degenerative processes in axons and other neuronal structures, including synaptic structures and dendritic processes, share similar mechanisms. To do this, we introduced both wild type and dominant negative Nmnat1 into cultured hippocampal neurons at 20 days in vitro (DIV 20), when their synaptic connections have formed and matured (Brewer et al. 1993), and examined their effects on axons as well as dendrites and synapses. As a conventional approach to visualize these neuronal structures, we also co-infected the hippocampal neurons with HSV expressing YFP. Our experiments revealed that most of the hippocampal neurons co-infected with HSV expressing YFP and wild type or mutant Nmnat1 did not show apoptotic signs at least 72 hr after viral infection, as indicated by the lack of staining with antibodies against active caspases and Hoechst 33258 nuclear staining. Moreover, neuronal NAD levels declined from 24-48 hr post-infection with viruses expressing dominant-negative Nmnat1 but not wild type Nmnat1 or YFP. Thus, we decided to focus our analysis on the first 60 hours post-infection.

To monitor changes in synaptic structures, we co-infected DIV20 neurons with HSV expressing YFP and wild type or the mutant Nmnat1, and quantified both spine density and the length and branching of axons and dendrites. As the density of dendritic spines has been considered a reliable indicator for synapse number (reviewed by Hering and Sheng, 2001), we performed high-resolution YFP imaging in neurons co-expressing YFP and Nmnat1 proteins at the indicated time points post-infection to measure the spine densities in these co-infected neurons. Dendrite length and branching patterns were analyzed using Sholl's method of regularly spaced concentric circles centered on the neuronal soma (Sholl, 1953). From these studies, we found that dendritic spine density decreased significantly 48 hr after infection with HSVs expressing dominant negative Nmnat1, but not wild type Nmnat1 or YFP alone, with no quantitative differences in dendrite length or complexity in these groups. However, at 60 hr post-infection, infection with HSVs expressing dominant-negative Nmnat1, but not wild type Nmnat1 or YFP, not only further decreased dendritic spine density, but also led to significant decreases in the numbers of intersections between dendrites and the Sholl circles at different distances from the soma in these neurons. In addition, in these neurons expressing dominant-negative Nmnat1, dendrites also appeared much thinner than controls, a phenomenon frequently found in aging and degenerating brains (Lolova et al., 1997, Anderson and Rutledge, 1996, Works et al., 2004). Such degenerative processes are unlikely to be secondary to cell death, as most of infected neurons did not show apoptotic signs at this stage, and the inclusion of caspase inhibitors did not change the course of neuronal degeneration. Sirt1 is also unlikely to be involved in such degenerative processes as including the Sirt1 activator resveratrol did not affect the changes induced by dominant-negative Nmnat1. Along the same line, introducing the sirt1 inhibitor sirtinol (from 50 to 100 μM) or nicotinamide (50-200 μM) alone did not induce significant changes in dendritic spine quantity or structure for up to 72 hr. However, both synaptic loss and neurite degeneration could be efficiently rescued by including NAD or methyl-pyruvate. These results together indicate that NAD decrease-triggered morphological changes in dendrites and synapses are mechanistically similar to Wallerian degeneration.

In the experiments described above, we also performed immunostaining with antibodies against the presynaptic marker bassoon, and found a decrease of positively-stained synaptic structures. However, due to the complex morphology of the axons that makes it extremely difficult to quantify the axonal length in DIV20 hippocampal neurons, we used DIV12 neurons, when the axonal projections are less complex, to examine the effects of dominant-negative Nmnat1 over-expression on axons and their synaptic terminals. Our data showed that dominant-negative Nmnat1, but not wild type Nmnat1 or YFP, induced a significant decrease in synaptic structures positively-stained with anti-bassoon antibodies starting from 48 hr post-infection. In addition, a shortening of average axonal length could also be seen from 60 hr post-infection in neurons expressing dominant-negative Nmnat1. Similar to the degenerative effects on dendrites and their spines, the changes in axons and presynaptic structures induced by dominant-negative Nmnat1 could also be rescued by NAD or methyl-pyruvate, but not Z-DEVD-fmk or resveratrol. Thus, our results indicate that the neuronal NAD decline triggered by the over-expression of dominant-negative Nmnat1 is sufficient to trigger a “dying-back” degeneration with initial synaptic loss and subsequent dendritic and axonal degeneration.

### EXAMPLE 8

### Age-Related NAD Levels Decrease in Mouse Brains

To explore the possible in vivo relevance of NAD metabolism to brain aging, we next examined whether there is a corresponding change in NAD levels from different regions of the mouse brain from different ages. In contrast to liver tissues, an age-dependent decrease of NAD levels was observed in the hippocampus and cerebellum, which could be detected as early as 7-10 months of age. Our results provide a molecular correlate for the selective vulnerability of certain brain regions to aging, such as the hippocampus and cerebellum (reviewed by Hof and Morrison, 2004).

### EXAMPLE 9

### Nicotinamide Reduces Neural Degeneration in Experimental Autoimmune Encephalomyelitis (EAE)

In this example, we show that nicotinamide reduces neural degeneration in an established mouse EAE model. EAE is an inflammatory, demyelinating disease that can be induced by immunizing animals against myelin antigens, and is an accepted animal model for multiple sclerosis (MS) as it has similarities based on both the histopathology and the clinical course of the affected animals. In methods adapted from Lo et al. (J Neurophysiol. 2003 November; 90(5):3566-71) we examine the effect of nicotinamide on axonal degeneration within the dorsal columns (cuneate fasciculus) and dorsal corticospinal tracts of the spinal cord. Using axonal counting, electrophysiology, and behavioral assessment after induction of EAE, we show that treatment with nicotinamide results in robust protection of the integrity of spinal cord axons, preservation of action potential conduction, and amelioration of neurological deficits.

Induction of EAE. Experiments are carried out in accordance with National Institutes of Health guidelines for the use of laboratory animals. C57/BL6 mice 6-10 wk of age are injected subcutaneously with 200 μl of an emulsion of 300 μg of rat myelin oligodendrocyte glycoprotein (MOG) 35-55 peptide (Keck Biotechnology Center, Yale University) in incomplete Freund's adjuvant (IFA, Sigma, St. Louis, Mo.) supplemented with 250-500 μg of Mycobacterium tuberculosis H37Ra (Difco, Detroit, Mich.). The MOG injection, with mycobacterium supplemented IFA, is repeated in the opposite flank 1 wk later. The mice also receive an injection of 250-500 ng pertussis toxin (Sigma) in 200 μL PBS ip immediately after the first immunization with MOG and then again 48 h later.

Nicotinamide treatment. Beginning on day 10 after the initial MOG injection, control and EAE-induced mice are fed pelleted mice chow incorporating nicotinamide (Sigma) to achieve an average daily consumption of 500 mg/kg nicotinamide. A separate group of EAE mice are fed the identical standard mice chow not containing nicotinamide. Serum nicotinamide levels are measured by HPLC (Chatzimichalakis et al, J Sep Sci. (2004) 27:1181-8) from two mice in each group on day 14 and on the day of either perfusion (days 27-28) or electrophysiological study (days 24-30). Mean serum nicotinamide levels are measured and are determined to be within the therapeutic range.

Mice are observed daily for clinical signs and scored on a 0-6 scale, with 0.5 gradations for intermediate scores, as follows: 0, normal without clinical signs; 1, flaccid tail; 2, abnormal righting reflex; 3, partial hindlimb paralysis; 4, complete hindlimb paralysis; 5, moribund; and 6, death. Nicotinamide-treated control mice do not display any pathological clinical signs. Untreated EAE mice manifest progressive clinical impairment exhibiting a score of 3-4 on day 27-28. In contrast, nicotinamide-treated EAE mice exhibit a less severe clinical course than untreated mice, exhibiting scores ranging from 1-2 on day 27-28.

Electrophysiological recordings. Fourteen adult male C57/BL6 mice are used for assessing spinal cord conduction. Untreated EAE (n=6) and nicotinamide-treated EAE (n=4) mice are studied between days 24 and 30 and age-matched nicotinamide-treated mice (n=4) are used as controls. Mice are anesthetized with halothane, and deep anesthesia is maintained with 1.1% halothane through a tracheal cannula by artificial ventilation (75-80 strokes/min, 0.7 ml/stroke) and verified by the absence of a withdrawal reflex to noxious peripheral pinch. Heartbeats (˜360 beats/min) and body temperature are carefully monitored. Mice are fixed in a stereotaxic apparatus (Kopf Instruments). A midline incision is made through the skin, a laminectomy is performed, and the dura is carefully cut to expose the lower thoracic and lumbar spinal cord regions. Silver wire electrodes (0.01-in diam, A-M Systems) insulated except at the tips are used for stimulation of dorsal column axons at L4-5 and recording of compound action potentials (CAP) at T11-12. Using stereotaxic measurements, the electrodes are positioned 8 mm apart at the surface of the midline spinal cord. Pancuronium bromide (0.3 mg/kg, Sigma) is injected ip to prevent further muscular contractions throughout the experiment. The signal from the recording electrode is amplified with filters set at 300-3,000 Hz (Dam 80, WPI) and collected (CED 1401, Cambridge Electronic Design), and a computer is used for data analysis (Spike 2 program, Cambridge Electronic Design). Single-current pulses (0.05 ms) are applied through a stimulus isolation unit (A365, WPI, and Master-8, AMPI) in increments (0.2, 0.3, 0.6, 0.8, and 1 mA). The amplitude of CAP is calculated as the value between the positive and negative peaks of the biphasic wave. The area of the CAP is calculated by rectifying the negative component (full-wave rectification using Spike 2 script software, Cambridge Electronic Design) and measuring its area. Conduction velocity is estimated by measuring the latency to the peak of the negative component of the supramaximal CAP at 8 mm, then moving the recording electrode to 9 mm and measuring latency; conduction velocities measured in this manner are within 5% of conduction velocities measured between the stimulating electrode and the recording electrode at 8 mm. At the end of each experiment, the dorsal columns and corticospinal tract are transected between stimulating and recording electrodes to confirm that a CAP could not be detected.

The average CAP amplitude and CAP area in the untreated EAE group is significantly smaller than in the nicotinamide-treated control group. The average threshold for evoking a CAP in the untreated EAE group is higher than in the nicotinamide-treated control group. In contrast, robust CAPs with a normal positive-negative configuration are observed in nicotinamide-treated EAE, with a threshold similar to that in controls, and the average CAP amplitude and area in the nicotinamide-treated EAE group is not significantly different from the nicotinamide-treated control group. Mean±SE conduction velocity in the EAE group is substantially slower than in controls but is maintained close to normal levels in nicotinamide-treated EAE. The data indicate preservation of conduction along spinal cord axons in nicotinamide-treated mice.

Immunocytochemistry. We measure the number of axons within the cervical spinal cord after staining them for neurofilaments. Mice are perfused with phosphate-buffered saline (PBS) and then with 4% paraformaldehyde in 0.14 M Sorensen's phosphate buffer. Spinal cords are carefully excised from the brain stem to the lumbar region and cryoprotected with 30% sucrose in PBS. The cervical enlargement is identified and then transected at the exact midpoint of the cervical enlargement to standardize a site along the longitudinal axis of the cord, ensuring that the same cervical spinal cord regions are analyzed for all conditions. Transverse sections are cut and incubated with antibodies against phosphorylated neurofilaments (SMI-31, 1:20,000; Sternberger Monoclonals, Lutherville, MD) and nonphosphorylated neurofilaments (SMI-32, 1:20,000) overnight at 4° C. on a rotating shaker. To count all axons, including axons with both phosphorylated and nonphosphorylated neurofilaments we incubate sections with SMI-31 and -32 antibodies in combination or individually with SMI-32, which has been used as a marker for demyelinated or damaged white matter axons, and with SMI-31. Sections reacted with SMI-31 and/or -32 are then incubated sequentially in PBS, rabbit anti-mouse IgG-biotin (1:1000, Sigma), PBS, avidin-HRP (1:1000, Sigma), PBS, and heavy metal-enhanced DAB (Pierce, Rockford, Ill.).

Image acquisition and analysis. Sections are examined with a Nikon E800 microscope equipped with a X100 oil objective, and digital images are captured with a Spot RT color camera (Spot Diagnostic Instruments, Sterling Heights, Mich.). Axonal densities are determined within preselected fields (500 μm² in area) at specific sites within the dorsal column (cuneate fasciculus), lateral column, ventral column, and dorsal corticospinal tract white matter regions, using the borders of the gray matter as consistent landmarks. Neurofilament stained axons are manually counted from each 500-μm² area using IPLab software (Scanalytics, Fairfax, Va.). The number of mice analyzed in each group are as follows: control, 4; control+nicotinamide, 5; EAE, 6; and EAE+nicotinamide, 5. Statistical analyses between control and experimental groups are performed using Student's t-test with Microsoft Excel software.

The difference in density of axons labeled with neurofilament antibodies within the dorsal corticospinal tract and within the dorsal columns (cuneate fasciculus) in untreated controls compared with nicotinamide-treated controls is not significant. There is significant loss of axons within the dorsal corticospinal tract and cuneate fasciculus of untreated EAE. The density of axons labeled with neurofilament antibodies in the corticospinal tract and in the cuneate fasciculus after 27-28 days of untreated EAE decreases substantially compared with nicotinamide-treated control mice. The data indicate that nicotinamide has a significant protective effect on dorsal corticospinal column and dorsal column (cuneate fasciculus) axons.

We have also demonstrated effective reduction of neural degeneration in EAE mice by administering the nicotinamide subcutaneously. In initial experiments, we administered nicotinamide daily starting from the day of immunization by MOG peptide, and found efficacy in both 125 mg/kg and 500 mg/kg treatment groups. We have also examined groups of mice wherein initiation of nicotinamide administration was delayed until 10 days after the immunization by MOG peptide (delayed treatment group), to better model clinical conditions wherein patients typically begin treatment after onset of pathogenic autoimmunity. We found this delayed treatment group also demonstrated reduced neural degeneration as compared with matched controls.

### EXAMPLE 10

### Nicotinamide Reduces Neural Degeneration in Amyotrophic Lateral Sclerosis (ALS)

In this example, we show that nicotinamide reduces neural degeneration in an established mouse (ALS) model, in a protocol adapted from Pombl et al. (FASEB J. 2003 April; 17(6):725-7).

Mice Mice expressing mutated human SOD1-G93A transgenic mice (heterozygous TgN-SOD1-G93A-1Gur; Gurney, et al., 1994, Science 264, 1772.1775) are obtained from the Jackson Laboratory (Bar Harbor, Me.). SOD1-G93A mice and wild-type (WT) control littermates are bred to generate groups of mice fed control diet or nicotinamide-enriched diets, respectively, beginning at 7 wk of age; WT control littermates provide control for specificity of phenotype (e.g. paralysis) and relative weight changes. Groups (n=6 per group, female) are sorted by genotype as described previously (by Gurney et al., 1994) and ultimately by phenotype. Mice are housed on a 12L:12D cycle and allowed ad libitum access to either nicotinamide-supplemented or plain NIH-07 formula cold press chow processed as described below.

Nicotinamide treatment In this study, we examine the role of chronic nicotinamide treatment in the feed of SOD1-G93A beginning at 7 wk of age with respect to the onset and progression of ALS symptoms: WT control littermates fed normal diet or nicotinamide controlled for specificity of changes. Nicotinamide is mixed directly with powdered rodent feed in a mixing drum into a homogeneous preparation, which is then formulated into rodent ½ inch feed pellets. All ingredients of the diet are from Zeigler Bros., Inc. (Garners, Pa.), and formulated diets are stored at 4° C. Mice (2.4 per cage) have access to food and water ad libitum, and diets are replenished regularly every 3 days.

On the basis of average food consumption, to achieve target dosages of about 1 and 5 mg per 35 g mouse (approximating 2 and 5 g/day human dosages), the treatment groups are fed a diet dose of 300 and 1500 mg nicotinamide per kg of diet, respectively. At time of death, necropsied mice do not reveal any detectable gross alteration of renal or intestinal abnormalities in either of the groups tested. During the course of the treatment with nicotinamide, mice are closely monitored for general health and neurologic status throughout the testing period. For example, no obvious differences between SOD1-G93A and WT littermates fed with nicotinamide or control diet (or WT controls) are found in lacrimation/salivation, eye blink test, ear twitch test, pupillary response, or whisker orienting reflex in either of the groups throughout the duration of this study. Assessment of muscle thickness and fat content by body condition scoring (BCS) is normal for all groups throughout the testing period (BCS=3), with the exception of both treated and untreated SOD1-G93A transgenics, which receive scores of 2 at the onset of bilateral hindlimb paralysis (see below).

Motor tests To assess the effect of nicotinamide on balance, coordination, and muscle strength, SOD1-G93A transgenic mice are tested on the accelerating Rotarod (7650 Ugo Basile Biol. Res. App.) and grid walking tests (Ma et al, 2001, Exp Neurol 169, 239-54). Mice are tested beginning at 82 days of age on both tasks three times per week until SOD1-G93A mice (fed control diet or nicotinamide feed) can no longer perform the tests. In all these studies, evaluators are blind to diet treatment at all stages of experimentation. The Rotarod test evaluates balance, coordination, and muscle strength. In this test, mice are placed onto a grooved cylinder (facing away from the experimenter) rotating at a predetermined speed that incrementally increases to a maximal rotation at 180 s; the time maintained on the rod by each mouse (latency) is then recorded (180 s max). A diminishing latency indicates declining performance and at values of 0 s is suggestive of severe muscular weakness and impaired coordination. Before testing, mice undergo a 1 wk training period wherein they are introduced to the apparatus and handled by the experimenter daily. Testing is conducted during the last 4 h of the day portion of the light cycle in an environment with minimal stimuli such as noise, movement, or changes in light or temperature. The maximum of 180 s is based on previous studies demonstrating that B6C3 mice can maintain 180 s latencies through to 200 days of age without any age associated or task rejection effects.

The grid walking test evaluates walking capacity as an index of muscle strength and coordination. This test is less physically demanding than the Rotarod test and provides a secondary measure that is testable for a brief period beyond that of the Rotarod. In the grid walking test, animals are placed into either end of a walled chamber (15 cm wide×60 cm long, 20 cm high walls) with a wire mesh bottom that is suspended 1 m above the floor. The animals are then tracked as they traverse the mesh grid of the chamber. The number of foot misses (entire paw and portion of limb pokes through the wire mesh) while crossing a distance of 60 cm is then recorded. A maximum value of 25 foot misses is assigned as an indicator of inability to complete the task. Similar to the Rotarod test, the maximum of 25 foot misses is based on previous studies demonstrating that B6C3 mice can maintain foot miss scores of (O) through to 200 days of age without any age associated or task rejection effects.

Time of death and collection of tissue for biochemical studies (nicotinamide concentration in spinal cord) At the onset of bilateral hindlimb paralysis (125-155 days of age), as determined by visual inspection, mice are killed by cervical dislocation. After being dissected, spinal cord samples (cervical) are dissected and snap frozen, pulverized on dry ice, and stored in −80° C. in aliquots for biochemical assessments. Blood (trunk) is also collected, and serum is obtained after 15 min of clotting and centrifugation in a clinical preparative centrifuge at 4° C.

Nicotinamide assessment Plasma samples are purified by a solid phase extraction procedure. Frozen plasma (100 μl) samples are thawed and diluted (1:20 vol/vol) in ice-cold loading buffer (50 mM Na₂HPO₄.12H₂O, pH 3), containing 2.5 μg/ml of 2.-(cyclohexyloxy)-4.-nitrophenyl methanesulphonanilide (NS398; Cayman). Samples are loaded on disposable C₁₈ columns (International Sorbent Technology, Isolute) primed with 1/1 vol of methanol and loading buffer. After being washed in 2 vol of loading buffer, samples are eluted in methanol (Sigma, St. Louis, Mo.). Samples are concentrated under nitrogen flow to dryness at 40° C. and then reconstituted in aliquots of mobile phase (100 μl; ethanol and acetonitrile; HPLC grade, Merck Darmastadt, Germany) and separated by HPLC. For assessment of nicotinamide in spinal cord, samples are homogenized (Polytron) in loading buffer and centrifuged at 10,000 g (10 m, 4° C.). As for the serum samples, the supernatants are concentrated and evaporated to dryness. The residues are reconstituted in 10 μl of mobile phase and separated by HPLC.

Statistics Statistical analysis is performed using the Prism software package (GraphPad Software Inc., San Diego, Calif.). Student's t test is used to test the significance between differences in mean values. ANOVA is used to compare greater than two groups, with Newman-Keuls multiple comparisons post hoc test to detect differences across groups. For all analyses, the null hypothesis is rejected at P<0.05.

Rotarod balance coordination behavioral assay SOD1-G93A mice fed control diet exhibit a mean onset of impairment in balance, coordination, and muscle strength, as assessed by the Rotarod motor test, at 108 days of age when compared with WT littermates. After day 108, motor impairment in SOD1-G93A mice progresses steadily as demonstrated by statistically lower latencies than that of prior trials through to 124 days of age. After 120 days of age, some animals can no longer perform the task and develop bilateral hindlimb paralysis within 1-2 wk thereafter (starting at 127 days of age).

Dietary supplementation with nicotinamide in SOD1-G93A transgenics significantly delays the onset of impairment in balance, coordination, and muscle strength, as assessed by Rotarod performance assay, and preserves motor functions through to 120 days of age. During this delay in motor impairment, nicotinamide fed SOD1-G93A mice perform statistically better than SOD1-G93A littermate mice fed control diet up to 120 days of age.

As with the nonsupplemented SOD1-G93A transgenics, after 120 days of age some animals can no longer perform the task (>180 s latencies) due to symptoms of hindlimb paralysis. WT control littermates group fed normal diet or diet with nicotinamide display optimum performance scores of 180 s throughout the testing period.

Grid walking test The grid walking test extends the information gathered by Rotarod assay by demonstrating impaired motor ability in a similar but independent assay. Impairment in grid walking in SOD1-G93A transgenic mice fed normal diet progresses steadily, showing that impairment of walking capacity is statistically higher than that of prior trials up to 124 days of age. Thus, the grid task extends motor measurements 2 days beyond that found by Rotarod testing. After 124 days of age, some animals can no longer perform the task and develop bilateral hindlimb paralysis within 1-2 wk thereafter. As done in the Rotarod studies, in order to maintain homogeneous group size, data collected after day 124 are not included in statistical comparisons.

Consistent with the Rotarod evidence, dietary supplementation with nicotinamide in the SOD1-G93A group starting at 7 wk of age significantly improves walking capacity through to 124 days of age. During the delay in motor impairment, nicotinamide fed SOD1-G93A mice perform statistically better than SOD1-G93A littermates fed control diet up to 124 days of age. However, similar to the Rotarod findings, after 124 days of age some animals can no longer perform the task and develop symptoms of hindlimb paralysis shortly thereafter.

WT groups fed normal diet or diet with nicotinamide exhibit normal stepping (foot miss scores of 0) without any foot misses throughout the testing period.

Bilateral hindlimb paralysis Behavioral data collection from either the Rotarod or grid walking tests is discontinued when animals receive scores of 0 s or 25 foot misses, respectively. Both SOD1-G93A mice fed normal diet and nicotinamide diet exhibit bilateral hindlimb paralysis at 127-157 days of age; at this time mice are killed for nicotinamide assessment in the spinal cord. No statistical differences between nicotinamide and control diet fed SOD1-G93A mice are found; however, there is a trend for delayed paralysis in the SOD1-G93A mice fed nicotinamide.

We have also demonstrated effective reduction of neural degeneration in ALS mice by administering the nicotinamide subcutaneously. In initial experiments, we administered nicotinamide daily starting at 10 weeks from birth, and have found efficacy in both 125 mg/kg and 500 mg/kg treatment groups, as measured by behavioral testing.

### EXAMPLE 11

### Nicotinamide Treatment of Relapsing Remitting Multiple Sclerosis (RRMS)

In this example, the safety and efficacy of nicotinamide therapy is evaluated in a clinical study adapted from a prior clinical trial entitled “A Multinational, Multicenter, Randomized, Double-Blind, Placebo-Controlled Study to Evaluate the Efficacy, Tolerability and Safety of 2 Doses (5 mg and 50 mg) of Glatiramer Acetate Orally Administered in Relapsing Remitting Multiple Sclerosis Patients” (see e.g. University of Pennsylvania Health System, Department of Neurology clinical trial information).

Nicotinamide and placebo are given orally as enteric-coated tablets. The subjects are monitored for resultant decrease in neural degeneration which is inferred primarily by reductions in the total number of relapses observed during treatment, and by other secondary objectives indicative of beneficial outcomes such as: other relapse variables (number of relapses treated with steroids); area under curve (AUC) of change in Kurtzke expanded disability status scale (EDSS) score; MRI variables such as: Number and volume of T1-Gd enhancing lesions, number and volume of T2 lesion, Number of new T1 Gd enhancing lesions (for the serial MRI cohort); and Tolerability and Safety profile.

For inclusion in the study, subjects must be males or nonpregnant, nonlactating females 18-50 years of age; have a diagnosis of clinically definite RRMS and disease duration of at least 6 months; have had at least one documented relapse within one year prior to study entry; and have an EDSS≦5.0 within 60 days before the first dose of study material.

The duration of the study will be one year. One-third of the study population will receive daily treatment with 2 g nicotinamide, one will receive 5 g nicotinamide, and the remaining one-third of the study population will receive placebo.

Subjects are monitored for adverse affects including increases in levels of liver aminotransferases and symptoms of hepatic dysfunction (McKenney et al, JAMA. 1994 Mar. 2; 271 (9):672-7).

### REFERENCES

Alcendor et al. (2004). Circ Res. 95, 971-980.
Ame et al. (2004). Bioessays. 26, 882-893.
Anderson R M, et al (2002). J Biol. Chem. 277, 18881-18890.
Anderson R M, et al. (2003). Nature. 423, 181-185.
Anderson B, and Rutledge V. (1996). Brain. 119, 1983-1990.
Araki T, et al (2004) Science 305, 1010-1013.
Berger F, et al. (2004). Trends Biochem Sci. 29, 111-118.
Bitterman K J, et al. (2002). J Biol. Chem. 277, 45099-45107.
Blander G, and Guarente L. (2004) Biochem. 73, 417-435.
Brewer G J, et al. (1993) J Neurosci Res. 35, 567-576.
Brunet A, et al (2004). Science 303, 2011-2015.
Bruzzone S, et al. (2001) FASEB J. 15, 10-12.
Bume J F, et al. (1996) J. Neurosci. 16, 2064-2073.
Chen S and Hillman D E. (1999). J. Neurocytol. 28, 187-196.
Coleman P D, and Flood D G. (1987). Neurobiol Aging. 8, 521-545.
Coleman, M., and Perry, V. H. (2002). Trends Neurosci 25, 532-538.
Conforti, L., et al (2000). Proc Natl Acad Sci USA 97, 11377-11382.
Deckwerth T L, and Johnson E M Jr. (1994). Dev Biol 165, 63-72.
Dillin A, et al (2002). Science 298, 2398-2401.
Duan H, et al (2003). Cerebral Cortex 13, 950-961.
Finn, J. T., et al. (2000) J Neurosci 20, 1333-1341.
Gallagher M, and Rapp P R. (1997). Annu Rev Psychol. 48, 339-370.
Garavaglia S, et al (2002). J Biol. Chem. 277, 8524-30.
Glass J D, et al (1993). JNeurocytol. 22, 311-321.
Grozinger C M, et alL. (2001). J Biol. Chem. 276, 38837-38843.
Guarente L, and Kenyon C. (2000). Nature 408, 255-262.
Gillingwater T H, et al (2002). J. Physiol. 543, 739-755.
Gillingwater T H, Ribchester R R. (2001). J. Physiol. 534, 627-639.
Gillingwater T H, and Ribchester R R. (2003). J Neurocytol. 32, 863-881.
Ha H C, and Snyder S H. (2000). Neurobiol Dis. 7, 225-239.
Hasty P, et al. (2003). Science. 299, 1355-1359.
Hedden T, and Gabrieli J D. (2004). Nat Rev Neurosci. 5, 87-96.
Hering H, and Sheng M. (2001). Nat Rev Neurosci. 2, 880-888.
Hof P R and Morrison J H. (2004). Trends Neurosci. 27, 607-613.
Howitz K T, et al (2003). Nature. 425, 191-196.
Imai S, et al. (2000). Nature. 403, 795-800.
Jacobs B, et al (1997). J Comp Neurol. 386, 661-680.
Kajander O A, et al (2002). Hum Mol Genet. 11, 317-24.
Kannurpatti S S, et al. (2004) Neurochem Int. 44, 361-369.
Kasischke K A, et al (2004). Science 305, 99-103.
Kenyon C. (2002). Cell 105, 165-168.
Kraus W L, and Lis J T. (2003). Cell 113, 677-683.
Koegl, M., et al (1999). Cell 96, 635-644.
Landry J, et al. (2000) Proc Natl Acad Sci USA. 97, 5807-5811.
Lee S S, et al. (2002). Nature Genetics 33, 40-48.
Lin S J, and. (2000). Science. 289, 2126-2128.
Lin S J, and Guarente L. (2003). Curr Opin Cell Biol. 15, 241-246.
Lolova I S, et al (1997) Mech Ageing Dev. 97, 193-205.
Lunn E R, et al. (1989). Eur J. Neurosci. 1, 27-33.
Lu T, et al. (2004). Nature 429, 883-891.
Luo J, et al. (2001). Cell 107, 137-148.
Mack, T. G., et al. (2001). Nat Neurosci 4, 1199-1206.
Magni G, et al (2004). Curr Med. Chem. 11, 873-885.
Marcotte P A, et al (2004) Anal Biochem. 332, 90-99.
Martin, G M and Loeb, L A (2004). Mice and mitochondria. Nature 429, 357-358.
McBurney M W, et al. (2003). Mol Cell Biol. 23, 38-54.
Mecocci P, et al (1993) Ann Neurol. 34, 609-616.
Micheli V, and Sestini S. (1997). Methods Enzymol. 280, 211-221.
Miledi R, and Slater C R. (1969). Proc R Soc Lond B Biol Sci. 174, 253-269.
Miledi R, and Slater C R. (1970). J. Physiol. 207, 507-528.
Motta M C, et al (2004). Cell 116, 551-563.
Morrison J H and Hof P R (1997). Science 278, 412-419.
Murphy C T, et al (2003) Nature 424, 277-283.
North B J, et al (2003). Mol Cell. 11, 437-444.
Onyango P, et al. (2003) Proc Natl Acad Sci USA. 99, 13653-13658.
Peters A, et al. (2000). J Comp Neurol. 419, 364-376.
Peters A. (2002). Prog Brain Res. 136, 455-465.
Picard F, et al (2004). Nature. 2004 429, 771-776.
Raff, M. C., et al (2002). Science 296, 868-871.
Raffaelli N, et al (2002). Biochem Biophys Res Commun. 297, 835-840.
Rongvaux A, et al. (2003). Bioessays. 25, 683-690.
Saigoh K, et al. (1999). Nat Genet. 23, 47-51.
Sala C, et al. (2001). Neuron. 31, 115-130.
Sandell J H, and Peters A. (2003). J Comp Neurol. 466, 14-30.
Saridakis V, et al. (2001). J Biol. Chem. 276, 7225-7232.
Schweiger M, et al. (2001). FEBS Lett. 492, 95-100.
Shibata, K, et al. (1986). Agri Biol Chem 50, 3037-3041.
Shibata K, et al. (1991). Adv Exp Med Biol. 294, 207-218.
Sievers C, et al (2003). Neurosci Res. 46, 161-169.
Sholl D A (1953). J. Anat. 87, 387-406.
Tissenbaum H A, and Guarente L. (2001) Nature. 410, 227-230.
Tsao J W, et al. (1994). Eur J. Neurosci. 6, 516-524.
Vaziri H, et al. (2001) Cell 107, 149-159.
Waller, A. (1850). Phil Trans R Soc Lond 140, 423-429.
Walsh D M, and Selkoe D J. (2004). Neuron 44, 181-193.
Wang M S, et al (2001a). Ann Neurol. 50, 773-779.
Wang M, et al. (2001b). Neurobiol Dis 8, 155-161.
West M J, and Gundersen H J. (1990). J Comp Neurol. 296, 1-22.
West M J et al. (1994). Lancet. 344, 769-772.
Winlow W and Usherwood P N. (1975). J Neurocytol. 4, 377-394.
Wong K K, et al (2003). Nature 421,643-648.
Works S J, et al (2004). Neurobiol Aging. 25, 963-974.
Yalowitz J A, et al (2004). Biochem J. 377, 317-326.
Yu C E, et al (1996). Science. 272, 258-262.
Yuan J, Yankner B A. (2000). Nature 407, 802-809.
Zhai Q, et al (2003). Neuron 39, 217-225.
Zhang Q, et al (2002). Science. 295, 1895-1897.
Zhang X et al. (2003). J Biol. Chem. 278, 13503-13511.
Zhou T et al. (2002). J Biol. Chem. 277, 13148-54.

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for reducing neural degeneration in a patient determined to be suffering from chronic neurodegeneration, the method comprising the steps of:
administering to the patient an effective amount of 3-pyridinecarboxamide (nicotinamide); and
detecting a resultant decrease in the neural degeneration, wherein the effective amount is 1 to 20 g/day and is administered daily for at least 90 days.

2. The method of claim 1 wherein the effective amount is 2 to 10 g/day.

3. The method of claim 1 wherein the nicotinamide is administered daily for at least 6 months.

4. The method of claim 1 wherein the nicotinamide is administered daily for at least 12 months.

5. The method of claim 1 wherein the nicotinamide is administered orally.

6. A method of doing business comprising promoting, marketing, selling or licensing a method for reducing neural degeneration in a patient determined to be suffering from chronic neurodegeneration, wherein the method for reducing neural degeneration in a patient comprises the steps of: administering to the patient an effective amount of 3-pyridinecarboxamide (nicotinamide), and detecting a resultant decrease in the neural degeneration; wherein the effective amount is 1 to 20 g/day and is administered daily for at least 90 days.
